(19) United States
(12) Patent Application Publication
(10) Pub. No.: US 2020/0061290 A1
(43) Pub. Date: Feb. 27, 2020

(54) PERFUSION SYSTEMS AND FLOW SENSORS FOR USE WITH PERFUSION SYSTEMS

(71) Applicant: onefusion AG, Zürich (CH)

(72) Inventors: Daniel Thorvaldsen, Zürich (CH); Jonas Gartmann, Zürich (CH)

(21) Appl. No.: 16/488,088

(22) PCT Filed: Feb. 12, 2018
(86) PCT No.: PCT/IB2018/050852

§ 371 Date: Aug. 22, 2019

Related U.S. Application Data
(60) Provisional application No. 62/462,426, filed on Feb. 23, 2017.

(51) Int. Cl. A61M 5/168; A61M 5/44

(52) U.S. Cl. CPC A61M 5/16886; A61M 2205/3368; A61M 5/44

(57) ABSTRACT

Flow sensing devices can be used for sensing flow rates of fluids flowing within a conduit. Systems can be used for controllably infusing a patient with a therapeutic medical fluid using the flow sensing devices for closed-loop control of the therapeutic medical fluid infusion flow rate. During the procedure, at least one calibration of the measuring process and the storage of at least one calibration value are performed. A control unit regulates the control valve or the syringe pump while using at least one measurement performed by the measuring device and taking into account at least one calibrated value towards the desired flow volume.

### CROSS-REFERENCE TO RELATED APPLICATIONS

This application claims the benefit of U.S. Provisional Application Ser. No. 62/462,426, filed Feb. 23, 2017. The disclosure of the prior application is considered part of (and is incorporated by reference in) the disclosure of this application.

### BACKGROUND

### 1. Technical Field

This disclosure relates to flow sensing devices that are used for sensing flow rates of fluids flowing within a conduit. The disclosure also relates to systems for controllably infusing a patient with a therapeutic medical fluid using the flow sensing devices for closed-loop control of the therapeutic medical fluid infusion flow rate.

### 2. Background Information

In customary perfusion devices, a container holding the perfusion liquid is positioned next to a drip container, to which is connected a perfusion conduit, specifically a perfusion tube. At the free end of the perfusion conduit is a perfusion needle, which is inserted into a vein of the patient who is to receive the perfusion. The perfusion conduit is fitted with a manually actuated control valve by which the flow velocity of the volume of perfusion liquid passing through the perfusion tube, and therefore the volume of perfusion liquid administered to the patient per time unit can be controlled.

During the use of such a perfusion device, the volume of perfusion liquid supplied to the patient per time unit is regulated by the control valve in the perfusion conduit, the objective being to meet the physiological needs of the patient. However, this procedure does not achieve the measurement of the flow velocity, nor is the course of the perfusion monitored. In a variation of this administration of a perfusion, a syringe pump is used instead of a perfusion container and a drip container. Here too, the flow velocity is not measured, nor is the course of the perfusion monitored.

### SUMMARY

This disclosure describes flow sensing devices that are used for sensing flow rates of fluids flowing within a conduit. The disclosure also describes systems for controllably infusing a patient with a therapeutic medical fluid using the flow sensing devices for closed-loop control of the therapeutic medical fluid infusion flow rate.

To be able to measure the volume of perfusion liquid administered to the patient per time unit, thereby permitting the flow velocity to be controlled and any changes to be detected in the flow of perfusion liquid through the perfusion conduit (e.g., changes due to blockages in the perfusion tube), this disclosure describes the use of a measuring device positioned along the perfusion conduit (e.g., abutting against but not affixed to the perfusion conduit) in order to measure the velocity of the perfusion liquid flowing through the perfusion conduit. The measured flow rate of the perfusion liquid can be shown on a display. This disclosure also describes a control unit to which the readings from the measuring device are sent, and by which the control/regulating valve is actuated to regulate the volume of perfusion liquid supplied to the patient per time unit.

In one aspect, this disclosure is related to a syringe pump device for dispensing a therapeutic medical fluid from a reservoir. Such a syringe pump device includes: (i) a housing; (ii) a drive assembly coupled to the housing and configured to pressurize the medical fluid within the reservoir such that the medical fluid is forced into an infusion tube in fluid communication with the reservoir; (iii) a controller coupled to the housing and in electrical communication with the drive assembly; and (iv) a flow rate sensor in electrical communication with the controller and configured to separably abut against an outer diameter of the infusion tube, the flow rate sensor comprising a heater and a single temperature sensor.

In another aspect, this disclosure is related to a method for controllably dispensing a medical fluid from a syringe pump. The method includes: (1) receiving, at a controller of the device, a flow rate input signal corresponding to a target flow rate of the medical fluid; (2) transmitting, by the controller and to a drive system of the device, a drive signal based on the flow rate input signal; (3) receiving, at the controller and from a flow rate sensor comprising a single temperature sensor, a flow rate measurement signal corresponding to a detected flow rate of the medical fluid; (4) comparing, by the controller, the target flow rate to the detected flow rate; and (5) modulating, by the controller and in response to the flow rate measurement signal, the drive signal.

In another aspect, this disclosure is directed to a device for dispensing a medical fluid from a syringe. Such a device includes: (a) a housing including structure for releasably coupling the syringe to the housing; (b) a drive assembly coupled to the housing and configured to drive dispensations of the medical fluid from the syringe into an infusion tube coupled to the syringe; (c) a controller coupled to the housing and in electrical communication with the drive assembly; and (d) a flow rate sensor comprising at least one temperature sensor in electrical communication with the controller and configured to abut against an outer diameter of the infusion tube while the syringe is coupled to the housing, the flow rate sensor not fixed to the infusion tube.

Such a device for dispensing a medical fluid from a syringe may optionally include one or more of the following features. The at least one temperature sensor may be one and only one temperature sensor. The at least one temperature sensor may be two or more temperature sensors. The at least one temperature sensor may be three or more temperature sensors.

In another aspect, this disclosure is directed to a method for operating a syringe pump to controllably dispense a medical fluid from a syringe. Such a method includes: (i) receiving, at a controller coupled to a syringe pump housing that is configured to releasably couple with the syringe, a flow rate input signal corresponding to a target flow rate of the medical fluid; (ii) transmitting, by the controller and to a drive system of the syringe pump, a drive signal based on the flow rate input signal, the drive system configured to drive movement of a plunger within the syringe such that the medical fluid flows from the syringe into an infusion tube; (iii) receiving, at the controller and from a flow rate sensor comprising at least one temperature sensor, a flow rate measurement signal corresponding to a detected flow rate of the medical fluid in the infusion tube, the flow rate sensor abutted against an outer diameter of the infusion tube while being unattached to the infusion tube; (iv) comparing, by the controller, the target flow rate to the detected flow rate; and (v) modulating, by the controller and in response to the flow rate measurement signal, the drive signal.

Such a method for operating a syringe pump to controllably dispense a medical fluid from a syringe may optionally include one or more of the following features. The at least one temperature sensor may be one and only one temperature sensor. The at least one temperature sensor may be two or more temperature sensors. The at least one temperature sensor may be three or more temperature sensors. In some cases, the detected flow rate changes in response to an elevation change of the syringe pump, and the modulating the drive signal includes compensating for the elevation change to adjust the flow rate to the target flow rate.

In another aspect, this disclosure is directed to a method for operating a syringe pump to controllably dispense a medical fluid from a syringe. Such a method includes: (a) transmitting, by a controller coupled to a syringe pump housing that is configured to releasably couple with the syringe, a first drive signal to a drive system of the syringe pump, the first drive signal corresponding to a first speed of the drive system; (b) receiving, at the controller and from a flow rate sensor comprising at least one temperature sensor, a flow rate measurement signal corresponding to an initially detected flow rate of the medical fluid in the infusion tube, the flow rate sensor abutted against an outer diameter of the infusion tube while being unattached to the infusion tube; and (c) in response to receiving the flow rate measurement signal, transmitting, by the controller, a second drive signal to the drive system, the second drive signal corresponding to a second speed of the drive system. The first speed is greater than the second speed.

Such a method for operating a syringe pump to controllably dispense a medical fluid from a syringe may optionally include one or more of the following features. The at least one temperature sensor may be one and only one temperature sensor. The at least one temperature sensor may be two or more temperature sensors. The at least one temperature sensor may be three or more temperature sensors.

In another aspect, this disclosure is directed to a control device for controlling a flow rate of a perfusion liquid flowing through a perfusion conduit. The control device includes: (1) a flow rate sensor configured to abut against the perfusion conduit, the flow rate sensor not fixed to the perfusion conduit; (2) a regulating valve configured to adjustably compress the perfusion conduit to regulate the flow rate of the perfusion liquid flowing through the perfusion conduit; and (3) a control unit in communication with the flow rate sensor and the regulating valve, the control unit configured to determine the flow rate of the perfusion liquid based on detecting a voltage drop across the heating element while a voltage supplied to the heating element is equilibrated with a voltage drop across the temperature sensor, the control unit configured to adjust the regulating valve based on the determined flow rate of the perfusion liquid. The flow rate sensor includes: a single temperature sensor, the temperature sensor detecting a temperature of the perfusion liquid; and a heating element spaced apart from the temperature sensor.

In another aspect, this disclosure is directed to a multi-modal flow rate sensor that includes: (1) a heating element; (2) a first temperature sensor disposed at a first side of the heating element; (3) a second temperature sensor disposed at a second side of the heating element, the second side of the heating element being opposite of the first side of the heating element; and (4) a third temperature sensor disposed at the second side of the heating element. The multi-modal flow rate sensor is operable in (i) a first flow-rate-sensing-mode and (ii) a second flow-rate-sensing-mode that differs from the first flow-rate-sensing-mode. The third temperature sensor is not used for first flow-rate-sensing-mode. The first and second temperature sensors are not used for the second flow-rate-sensing-mode.

In another aspect, this disclosure is directed to a control device for controlling a flow rate of a perfusion liquid flowing through a perfusion conduit. The control device includes: (a) a flow rate sensor configured to abut against the perfusion conduit (the flow rate sensor not fixed to the perfusion conduit); (b) a regulating valve configured to adjustably compress the perfusion conduit to regulate the flow rate of the perfusion liquid flowing through the perfusion conduit; and (c) a control unit in communication with the flow rate sensor and the regulating valve. The control unit is configured to determine the flow rate of the perfusion liquid in a first flow-rate-sensing-mode using a difference in temperatures detected by the first and second temperature sensors. The control unit is configured to determine the flow rate of the perfusion liquid in a second flow-rate-sensing-mode based on a detected voltage drop across the heating element while the voltage supplied to the heating element is equilibrated with the voltage drop across the third temperature sensor. The control unit is configured to adjust the regulating valve based on the determined flow rate of the perfusion liquid. The flow rate sensor includes a single temperature sensor (the temperature sensor detecting a temperature of the perfusion liquid), and a heating element spaced apart from the temperature sensor.

In another aspect, this disclosure is related to a device for determining a flow rate of a perfusion liquid flowing through a perfusion conduit. The device includes: a flow rate sensor configured to abut against the perfusion conduit (the flow rate sensor not fixed to the perfusion conduit) and a control unit in communication with the flow rate sensor. The control unit is configured to determine the flow rate of the perfusion liquid based on a time difference between a first time when a voltage is applied to the heating element and a second time when a change in resistance of the temperature sensor corresponding to the voltage applied to the heating element is detected. The flow rate sensor includes: a single temperature sensor; and a heating element spaced apart from the temperature sensor.

In another aspect, this disclosure is directed to a control device for controlling a flow rate of a perfusion liquid flowing through a perfusion conduit. The control device includes: (i) a flow rate sensor configured to abut against the perfusion conduit (the flow rate sensor not fixed to the perfusion conduit); (ii) a regulating valve configured to adjustably compress the perfusion conduit to regulate the flow rate of the perfusion liquid flowing through the perfusion conduit; and (iii) a control unit in communication with the flow rate sensor and the regulating valve. The control unit is configured to determine the flow rate of the perfusion liquid based on a time difference between a first time when a voltage is applied to the heating element and a second time when a change in resistance of the temperature sensor corresponding to the voltage applied to the heating element is detected. The control unit is configured to adjust the regulating valve based on the determined flow rate of the perfusion liquid. The flow rate sensor includes: a single temperature sensor; and a heating element spaced apart from the temperature sensor.

In another aspect, this disclosure is directed to a device for determining a flow rate of a perfusion liquid flowing through a perfusion conduit. The device includes: (a) two flow rate sensors configured to abut against the perfusion conduit (the flow rate sensors not fixed to the perfusion conduit); and (b) a control unit in communication with the flow rate sensors. The control unit is configured to determine the flow rate of the perfusion liquid using a time difference between detected temperature rises of the first temperature sensor and the second temperature sensor. Each of the flow rate sensors include: a first temperature sensor; a second temperature sensor; and a heating element between the first and second temperature sensors.

To perform an exact measurement of the volume of perfusion liquid flowing through the perfusion conduit per time unit, it is necessary to calibrate the measuring process, i.e., to detect the parameters that determine the measuring process in terms of the volume of perfusion liquid flowing through the perfusion conduit, and to store these parameters. For this purpose, every perfusion conduit, in particular every perfusion tube, should ideally be equipped with a storage device in which the calibration data for that particular perfusion tube are saved so that they are available to facilitate accurate measurements of the flow velocity when the particular perfusion conduit is being used. However, since perfusion conduits, especially perfusion tubes, are used only once for a perfusion and then disposed of, this would lead to insupportably high costs.

The systems and methods described herein therefore have an objective of creating an economically feasible procedure to operate a perfusion device that would enable a precise volume of perfusion liquid to be administered to a patient per time unit. As described further below, this is achieved by performing at least one calibration of the measuring process (e.g., drop detector or syringe pump) and by measuring at least one volume of the perfusion liquid flowing through the perfusion conduit per time unit, (where the control valve or syringe pump is regulated during the perfusion by using at least one measurement obtained by the measuring device), while taking into account at least one calibrated value for the intended flow volume.

It is preferable to perform several calibrations successively while storing the calibrated values. In so doing, at least one calibration can be performed before or when the perfusion begins. In addition, calibrations can also be performed during the perfusion or throughout the entire duration of the perfusion, with at least some of the calibrated values being used to regulate the perfusion.

Since the measuring devices described herein are extremely sensitive, such measuring devices can also advantageously be used as a drop detector to measure at least one volume of the perfusion liquid flowing through the infusion conduit per time unit.

If a malfunction occurs during the perfusion, it can advantageously be detected using the systems and methods described herein, and then displayed by the regulating unit. In addition, the perfusion can be stopped by the regulating unit if necessary.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is perspective view of a cross-section of a perfusion conduit that has an example flow sensing device abutted against the conduit (but not affixed to the conduit).

FIG. 2 is an example schematic circuit diagram that can be used to operate the flow sensing device of FIG. 1.

FIG. 3 is a chart depicting the output signal of the flow sensing device of FIG. 1.

FIG. 4 is perspective view of a cross-section of a perfusion conduit that has another example flow sensing device abutted against the conduit (but not affixed to the conduit).

FIG. 5 is an example schematic circuit diagram that can be used to operate the flow sensing device of FIG. 4.

FIG. 6 is a chart depicting the output signal of the flow sensing device of FIG. 4.

FIG. 7 is perspective view of a cross-section of a perfusion conduit that has another example flow sensing device abutted against the conduit (but not affixed to the conduit).

FIG. 8 is a time-based plot showing the flow sensing device of FIG. 7 outputting a heat pulse and detecting of the heat pulse.

FIG. 9 is another view of the arrangement of FIG. 7.

FIG. 10 is a time-based plot showing the flow sensing device of FIG. 9 outputting a series of heat pulses and detecting of the series of heat pulses.

FIG. 11 is perspective view of a cross-section of a perfusion conduit that has another example flow sensing device abutted against the conduit (but not affixed to the conduit).

FIG. 12 is a time-based plot showing the flow sensing device of FIG. 11 outputting a heat pulse and detecting of the heat pulse.

FIG. 13 is perspective view of a cross-section of a perfusion conduit that has another example flow sensing device abutted against the conduit (but not affixed to the conduit).

FIG. 14 is a time-based plot showing the flow sensing device of FIG. 13 outputting a heat pulse and detecting of the heat pulse.

FIG. 15 is perspective view of a cross-section of a perfusion conduit that has another example flow sensing device abutted against the conduit (but not affixed to the conduit).

FIG. 16 is perspective view of a cross-section of a perfusion conduit that has another example flow sensing device abutted against the conduit (but not affixed to the conduit).

FIG. 17 is an example schematic circuit diagram that can be used to operate the flow sensing device of FIG. 16.

FIG. 18 is a chart depicting the output signal of the flow sensing device of FIG. 16.

FIG. 19 is a perspective view of an example syringe pump system in accordance with some embodiments.

FIG. 20 is a schematic representation of the example syringe pump system of FIG. 19.

FIG. 21 is a schematic representation of a portion of the example syringe pump system of FIG. 19.

FIG. 22 is a time-based plot illustrating the time delay of the infusion fluid because of inherent system compliances of the example syringe pump system of FIG. 19.

FIG. 23 is a time-based plot illustrating that the time delay of the infusion fluid (as illustrated in FIG. 22) can be reduced in accordance with the concepts provided herein.

FIG. 24 is a perspective view of a patient receiving an infusion from the example syringe pump system of FIG. 19.

FIGS. 25 and 26 are time-based plots illustrating that boluses associated with a change in elevation of the syringe pump system of FIG. 19 can be attenuated in accordance with the concepts provided herein.

FIG. 27 is a perspective view of an example infusion system.

FIGS. 28-31 are various views of an example infusion control system that includes a regulating valve and a flow sensing device.

Like reference numbers represent corresponding parts throughout.

### DETAILED DESCRIPTION

This disclosure describes flow sensing devices that are used for sensing flow rates of fluids flowing within a conduit. The disclosure also describes systems for controllably infusing a patient with a therapeutic medical fluid using the flow sensing devices for closed-loop control of the therapeutic medical fluid infusion flow rate.

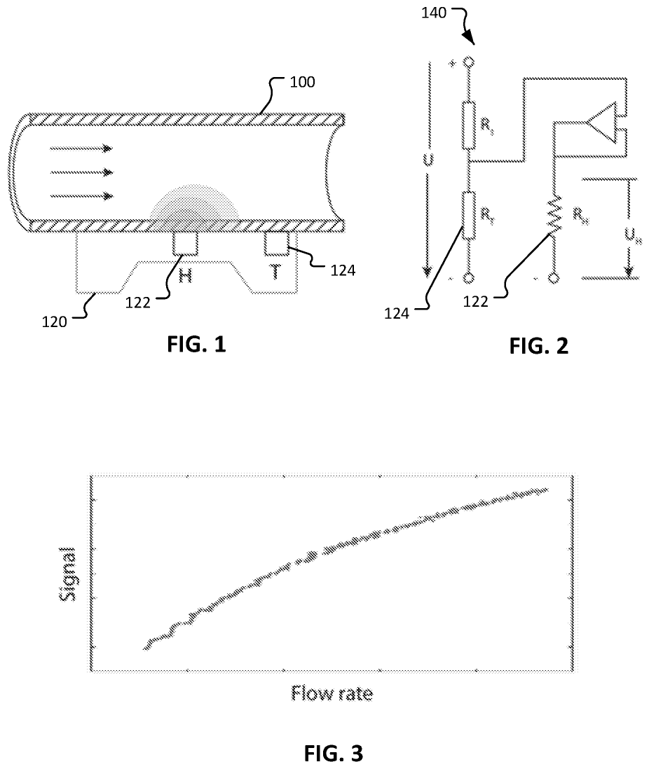

Referring to FIGS. 1-3, this disclosure includes a description of, inter alia, apparatuses to measure and/or control the flow rate of a fluid inside tubing (e.g., a plastic tube) using a flow rate sensor that is pressed against (abutted against), but not affixed to (i.e., readily separable from), the tubing. In some embodiments, a regulating valve and a controller are included in a system with the flow rate sensor to provide the facilities to modulate the fluid flow rate based on the flow rate measurement provided by the flow rate sensor. The inventive concepts described herein are typically intended to be used with an infusion set for infusion therapy, however, various other implementations are also envisioned and are within the scope of this disclosure.

For some implementations described herein that measure and control the flow of a therapeutic medical fluid (e.g., a drug, medicant, saline, etc.) within a perfusion conduit, a flow sensor 120 is provided that merely abuts against the outside of the perfusion conduit 100, as shown in FIG. 1. That is, the flow sensor 120 is not affixed to the perfusion conduit 100.

In the depicted embodiment, the flow sensor 120 includes a heating element 122, and a temperature sensor 124 positioned at a distance away from the heating element 122. The flow sensor 120 is positioned to measure the temperature of the fluid within the perfusion conduit 100. The heater 122 is wired in a circuit to be kept at a constant temperature above the fluid within the perfusion conduit 100.

Now also referring to FIG. 2, one possible circuit 140 for operating flow sensor 120 is shown. In the circuit 140, the supply voltage UH of the heating element 122 is kept at the level of the voltage drop above the temperature sensor 124. This way, if there are temperature changes within the medium, the flow sensor 120 is less dependent on them.

The flow of the perfusion liquid causes a temperature change of the heater 122, the faster the flow the more the heater 122 is cooled down. The voltage drop UH across the heater 122 can thus be used to measure the flow rate within the tubing 100. Such a response curve is shown in FIG. 3, showing the voltage drop (the Y-axis) across the flow sensor 120 at different flow rates (the X-axis).

In some cases, the signal output by the flow sensor 120 is used in a closed-loop control system to regulate the flow within a perfusion conduit using a control valve. In some embodiments, such a control valve is designed to adjustably squeeze or pinch the tubing, thus modifying the cross-sectional shape/area of the conduit to thereby modulate the pressure drop created by the control valve (and the flow of the fluid in the tubing in result). At a prevailing flow within the tubing (e.g., driven by gravity or a mechanical driving mechanism), a control unit will receive signals from the flow sensor 120 and control the valve accordingly (e.g., to a set point flow rate).

In some embodiments, the system can be used to control the therapeutic medical fluid flow rate to a set point input by the user of the system. For example, the system can be used to regulate the flow of a drug through a perfusion conduit to a patient, and to correct the flow rate if it differs from a target flow rate.

In some embodiments, the system can also be used to detect failures in the infusion therapy immediately. Those failures could be, e.g., a kinking in the tubing that results in an occlusion and fully or partially prevents the drug from reaching the patient at the desired flow rate. Another failure could be an occlusion inside the vein of the patient, also fully or partially preventing the drug from reaching the patient at a desired flow rate.

In some cases, the signal from the flow rate sensor 120 (using a heater 122 and a single temperature sensor 124) is effected by one or more other parameters beside the flow rate within the infusion tubing 100. Such parameters may include, for example, the wall thickness of the tubing 100, the medium flowing within the tubing 100, and the like. To compensate for such influences, in some cases one or more other flow rate measuring devices are used to calibrate the flow signal from the flow sensor 120. In one such example, a drop detector is used to calibrate the flow sensor 120. That is, a drop detector (which measures flow rate based on counting drops of known drop size) is used to detect and count the falling of drops within a drip chamber that is fluidly coupled to infusion tubing 100.

In some embodiments, based on the descriptions provided above in reference to FIGS. 1-3, an inventive control device/system is described herein for controlling a flow rate of a perfusion liquid flowing through a perfusion conduit 100. The control device includes: a flow rate sensor 120 configured to abut against the perfusion conduit 100 (the flow rate sensor 120 is not fixed to the perfusion conduit), a regulating valve configured to adjustably compress the perfusion conduit 100 to regulate the flow rate of the perfusion liquid flowing through the perfusion conduit 100, and a control unit in communication with the flow rate sensor 120 and the regulating valve. The flow rate sensor 120 includes a single temperature sensor 124 (one and only one temperature sensor 124). The temperature sensor detects a temperature of the perfusion liquid. The flow rate sensor 120 also includes a heating element 122 spaced apart from the single temperature sensor 124. The control unit is configured to determine the flow rate of the perfusion liquid based on detecting a voltage drop across the heating element 122 while a voltage supplied to the heating element 122 is equilibrated with a voltage drop across the temperature sensor 124. The control unit is also configured to adjust the regulating valve based on the determined flow rate of the perfusion liquid.

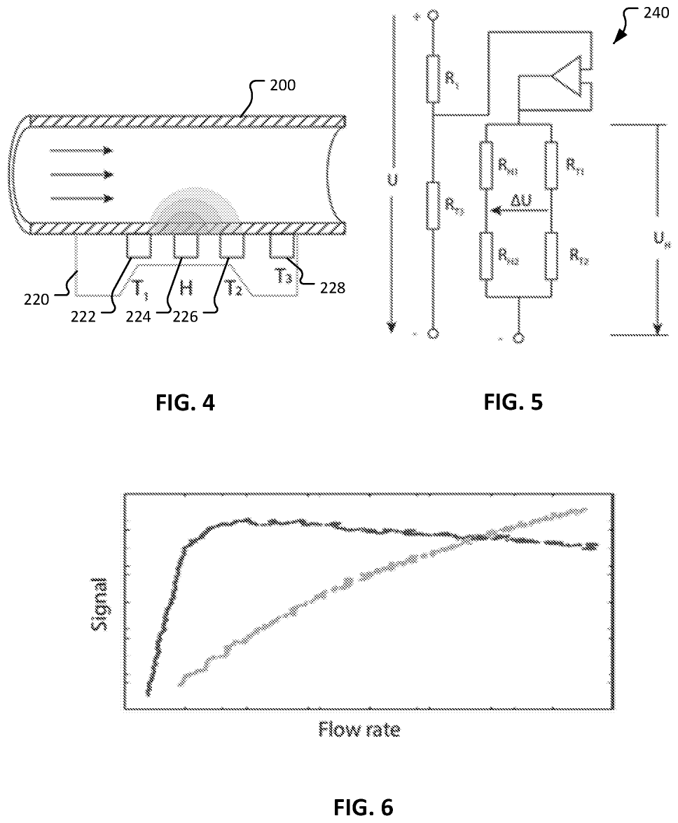

Referring to FIGS. 4-6, this disclosure also describes a flow sensor to measure the flow rate within a conduit such as a perfusion tube across a broad range of flow rates by combining two measurement principles within a single flow sensor device.

As shown in FIG. 4, a flow sensor 220 is designed to abut, while not being affixed to, a perfusion conduit 200 in which there is a prevailing flow regime (i.e., a flow of a liquid such as an therapeutic medical fluid). The flow sensor 220 comprises a heating element 224, which is placed between two temperature sensors 222 and 226, and a third temperature sensor 228, which is placed downstream from the heating element 224.

This flow sensor 220 can be operated in two different flow-detection modes. In the first operation mode, the heating element 224, which is positioned to abut the perfusion conduit, heats up the fluid within the perfusion conduit 200. The prevailing flow regime within the perfusion conduit 200 causes the heat to be transported downstream, thus causing a temperature difference between the upstream temperature sensor 222 and the downstream temperature sensor 226. This temperature difference can be used to measure the flow rate of the liquid within the perfusion conduit 220.

In the second operation mode, the third temperature sensor 228 measures the temperature of the liquid within the perfusion tubing 200. The heater 224 is then operated at a constant temperature above the temperature of the liquid flowing within the perfusion tubing 200. The prevailing flow regime within the tubing 200 causes the heater 224 to be cooled down. This cooling affects the resistance of the heater 224 and the voltage drop across the heater 224, which is used to measure the flow rate within the perfusion tubing 200.

The first measurement principle allows determining the flow direction of the fluid, since the temperature difference between the two temperature sensors 222 and 226 can be either positive or negative. Also the first measurement principle has a good signal-to-noise ratio at low flow rates. On the downside, the first measurement principle has a turning point, at which the temperature difference between the two temperature sensors 222 and 226 increases for low flow rates. After crossing a certain threshold, the temperature difference between the two temperature sensors 222 and 226 decreases again with increasing flow rates. This makes the first measurement principle not well-suited for higher flow rates.

The second measurement principle cannot determine the direction of the flow regime and has a worse signal-to-noise ratio than the first measurement principle at low flow rates. On the upside, it has a better signal-to-noise ratio at higher flow rates and no turning point behavior of the signal.

The flow sensor device 220 uses both the first and the second measurement principles on one flow sensor chip to combine the two flow measurement principles to measure the flow rate within an infusion tubing 200 across a wider range of flow rates and determine the flow direction within the tubing 200. One possible embodiment of an electronic circuit of the measurement principle is shown in FIG. 5. Such an embodiment may comprise a bridge circuit with four resistors, two resistors are used as heaters, one is used as temperature sensor, downstream from the heater, and one is used as temperature sensor, upstream from the heater. The voltage supply of the bridge circuit is kept at the level of a voltage drop of another temperature sensing resistor. The bridge circuit is used to determine the temperature difference between the upstream temperature sensor 222 and the downstream temperature sensor 226 from the heater element 224. The supply voltage to the bridge circuit is kept at the same level as the voltage drop across a third temperature sensor 228, and is measured to determine the flow rate, using the second measurement principle.

Measuring with both measurement signals in the same circuit has the advantage of both principles without a much higher complexity on the circuit, it has a good signal-to-noise ratio at both low and higher flow rates, overcomes the reversal point problem, is bi-directional and less dependent on media temperature changes. FIG. 6 shows a sample measurement of both measurement principles at different flow rates.

By combining this flow sensor 220 with a regulating valve, designed to squeeze the perfusion conduit, thus modifying the pressure drop across the valve and thus the flow rate within the tubing, and a control unit, receiving a signal corresponding to the flow rate from the flow rate sensor and controlling the valve, the flow rate within a perfusion conduit can be controlled using a closed-loop control.

In some cases, the output signal of the flow sensor 220 is used in a closed-loop control system to regulate the flow within a perfusion conduit using a control valve. In some embodiments, such a control valve is designed to adjustably squeeze or pinch the tubing, thus modifying the cross-sectional shape/area of the conduit to thereby modulate the pressure drop created by the control valve (and the flow of the fluid in the tubing in result). At a prevailing flow within the tubing (e.g., driven by gravity or a mechanical driving mechanism), a control unit will receive signals from the flow sensor 220 and control the valve accordingly (e.g., to a set point flow rate).

The flow sensor 220 can be used in a system with a control unit in communication with the flow rate sensor 220 and with a flow regulating valve designed to adjustably squeeze or pinch the tubing to which the flow sensor 220 is abutted (but not affixed). The control unit can be configured to determine the flow rate of the perfusion liquid in a first flow-rate-sensing-mode using a difference in temperatures detected by the first and second temperature sensors 222 and 226. The control unit can additionally be configured to determine the flow rate of the perfusion liquid in a second flow-rate-sensing-mode based on a detected voltage drop across the heating element 224 while the voltage supplied to the heating element 224 is equilibrated with the voltage drop across the third temperature sensor 228. The control unit can also be configured to adjust the regulating valve based on the determined flow rate of the perfusion liquid.

In some embodiments, the system can be used to control the therapeutic medical fluid flow rate to a set point input by the user of the system. For example, the system can be used to regulate the flow of a drug through a perfusion conduit to a patient, and to correct the flow rate if it differs from a target flow rate.

The multi-modal flow rate sensor 220 includes a heating element 224; a first temperature sensor 222 disposed at a first side of the heating element 224; a second temperature sensor 226 disposed at a second side of the heating element 224 (the second side of the heating element being opposite of the first side of the heating element 224); and a third temperature sensor 228 disposed at the second side of the heating element 224. As described above, the multi-modal flow rate sensor 222 is operable in (i) a first flow-rate-sensing-mode and (ii) a second flow-rate-sensing-mode that differs from the first flow-rate-sensing-mode. The third temperature sensor 228 is not used for first flow-rate-sensing-mode. The first and second temperature sensors 222 and 226 are not used for the second flow-rate-sensing-mode.

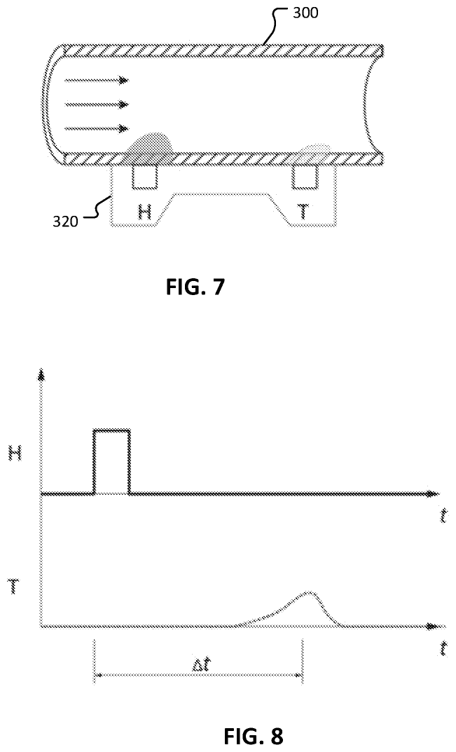

Referring to FIGS. 7-15, this disclosure also describes a procedure to measure the flow rate within a system comprising a plastic tube, a heater, at least one temperature sensor, and a control unit configured to determine the flow rate using the signals sent to the heater and the signals received from the temperature sensor. In some embodiments, the heater and temperature sensor are combined in a flow rate sensor that is abutting, but not affixed to, a conduit.

The heater, introduces a certain amount of heat to the fluid within the conduit and increases the temperature in a local part of the fluid (i.e., essentially the fluid adjacent to the heater). By the fluid flow prevailing inside the tubing, this heated part of the fluid is moved downstream. Once the heated fluid reaches the temperature sensor, the temperature at the sensor increases, registering the arrival of the heated fluid. The time difference between the heater pulse and the temperature increase at the temperature sensor is related to the fluid velocity and the fluid flow rate within the tubing. If the distance between the heater and the temperature sensor and the cross-section of the tubing are known, the prevailing volume flow can be calculated, using those parameters. This principle is shown in FIGS. 7 and 8 (where the heater is “H” and the temperature sensor is “T”). FIG. 8 shows a time difference Δt between the time at which the heat pulse is delivered to the fluid by the heater H and the time at which the heated fluid reaches the temperature sensor T.

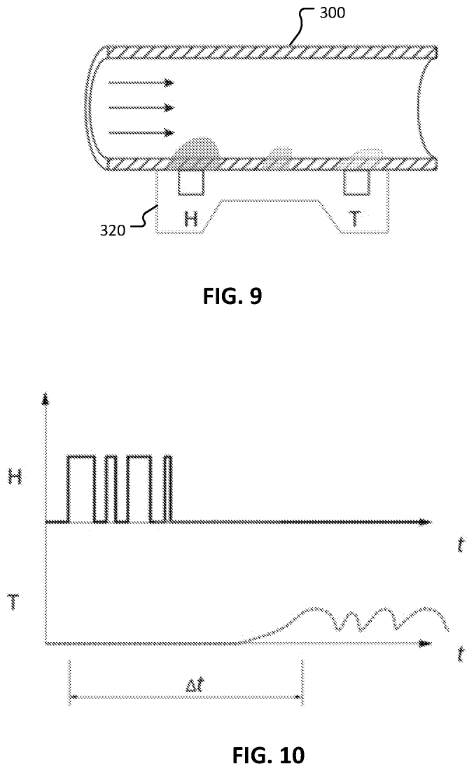

Additionally, as illustrated in FIGS. 9 and 10, instead of using a single pulse, there can be multiple heat pulses delivered to the fluid by the heater H. In some embodiments, a randomized series of heat pulses can be delivered by at the heater H. Such a randomized series results in a certain response at the temperature sensor T. Since the flow regime is not constant across the cross-section of the tubing, but parabolic, and due to a heat conduction through the fluid, the signal at the temperature sensor T might not be distinct. There, one possible mathematical operation to determine the time difference might be a cross-correlation of the two signals. The time shift, at which the resulted cross-correlation is at the maximum is the time of flight of the heated up fluid. This “footprint” of the heater signal is illustrated in FIG. 10.

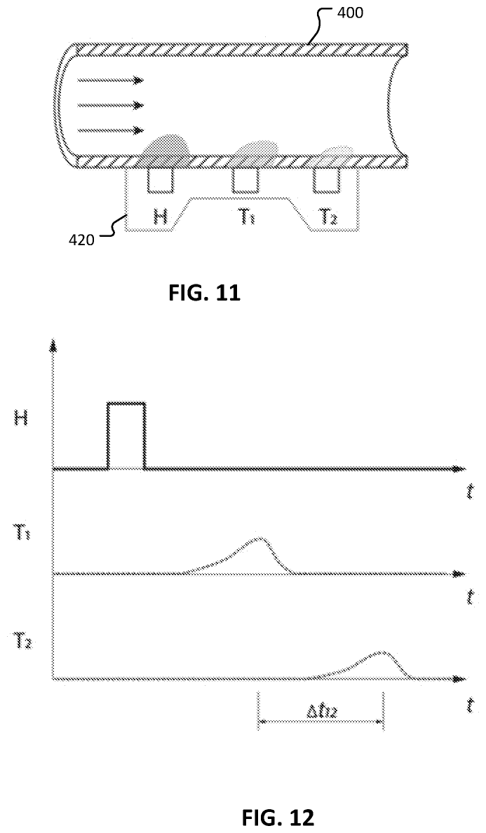

Also, instead of using a single temperature sensor, there could be two or more temperature sensors downstream of the heater (wherein the heater and temperature sensors are included in a flow sensor device). Instead of measuring the time difference between the heat pulse (input) and the temperature sensor response (of a single temperature sensor), the time difference between the two temperature sensor responses can be measured. An illustration of this principle is shown in FIGS. 11 and 12. FIG. 12 shows a time difference Δt₁₂ between the time at which the heated fluid is detected by the first temperature sensor T₁ and the time at which the heated fluid is detected by the second temperature sensor T₂. Again, a randomized pulse can be used together with cross-correlation of the responses to detect the time of flight between the temperature sensors T₁ and T₂.

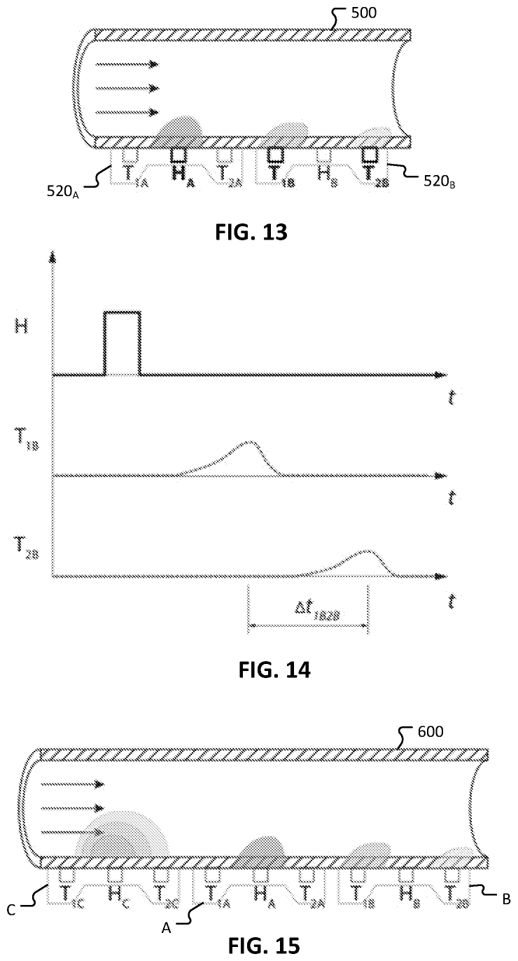

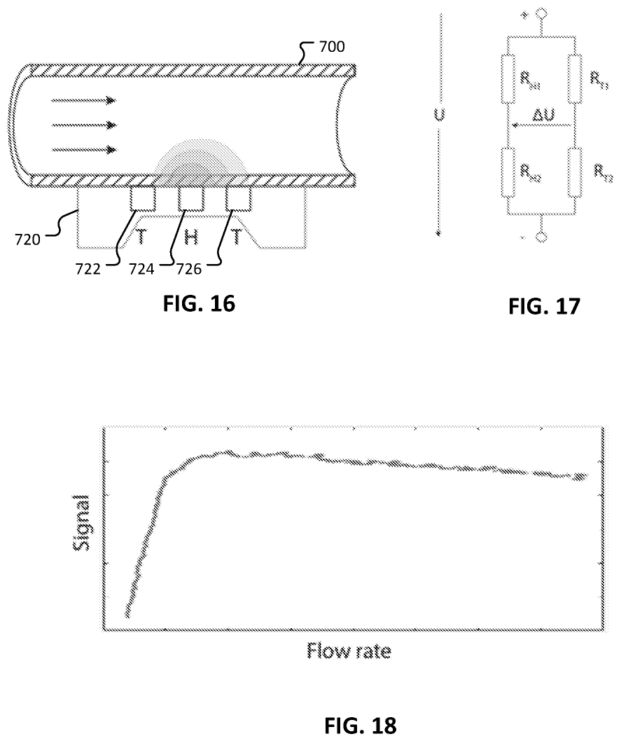

Instead of using a single flow sensor chip (containing both a heater and a temperature sensor) the heater and the temperature sensor(s) used for measuring the flow can be on separate sensor chips (e.g., in separate flow sensor devices). One possible implementation is illustrated in FIG. 13. The system of FIG. 13 includes two sensor chips 520_{A} and 520_{B}. Sensor 520_{A} includes a heater element HA disposed between two temperature sensors T_{1A} and T_{2A}. Sensor 520_{B} includes a heater element H_{B} disposed between two temperature sensors T_{1B} and T_{2B}. The two sensor chips 520_{A} and 520_{B} are placed next to each other, both designed to abut the infusion tubing 500 (without being affixed to the infusion tubing 500). In the depicted embodiment, the first sensor chip 520_{A} is used as a heater, i.e. the heater HA in the middle generates heat pulses. On the second sensor chip 520_{B} abutting the infusion tubing 500, the first and second temperature sensors T_{1B} and T_{2B} each detect the increase in temperature of the fluid. As shown in FIG. 14, the time difference Δt_{1B2B} between the detected fluid temperature increase at the two temperature sensors T_{1B} and T_{2B} equals the time of flight which can be used to calculate the fluid flow rate when the cross-sectional diameter (area) of the tube 500 is known. Again, randomized heat pulses might be used and a cross-correlation to determine the time-of-flight. The distance between the two temperature sensors T_{1B} and T_{2B} and the cross-sectional area of the tubing 500 determine the correlation between the time of flight and the volume flow of the fluid.

These time-of-flight measurement concepts can also be used in combination with one or more other flow measurement methods, e.g., a calorimetric flow sensor, as depicted in FIG. 15. In the depicted embodiment, the calorimetric sensor C (comprising heater H_{C} and temperature sensors T_{1C} and T_{2C}) is the same type of flow sensor chip as the two sensor chips A and B that are used for the time-of-flight measurement. Each one of the three flow sensors A, B, and C comprise a heating element and two temperature sensors (one temperature upstream of the heating element and one temperature sensor downstream of the heating element).

The temperature difference between the two temperature sensors T_{1C} and T_{2C} is used to measure the flow rate of the fluid within the perfusion tubing 600. This flow sensor C provides a fast, real-time signal, which is dependent on many variables, such as the wall thickness of the tube 600, the medium of the fluid, etc. The time-of-flight measurement (using flow sensors A and B in this example) provides a slower, but more independent signal. Therefore, the time-of-flight measurement can be used to calibrate the faster calorimetric signal to provide a fast, real-time, independent signal. Instead of the calorimetric principle, the first sensor chip C could also use the anemometric or the constant-temperature-anemometric principle.

In another embodiment, the flow sensor C comprises a heating element H_{C} and a single temperature sensor T_{C}. The temperature sensor T_{C} measures the temperature of the fluid within the perfusion conduit. The cooling of the heating element H_{C}, i.e. the voltage drop across the heater H_{C}, is used to measure the flow rate within the perfusion conduit. In another embodiment, a multi-modal flow sensor is used, comprising a heating element and three temperature sensors, operating in two flow-rate-sensing-modes.

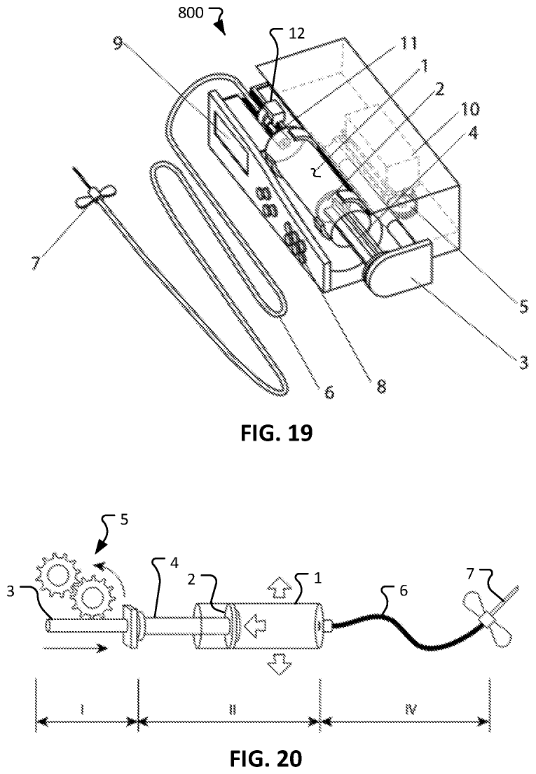

Referring to FIGS. 19 and 20, this disclosure also describes innovative device and systems for delivering an therapeutic medical fluid using a syringe pump. As described further below, the devices and systems described herein mitigate some shortcomings of some conventional syringe pump devices, such as compliance in the system. Such system compliance can lead to a reduction of the precision of controlling an infusion.

The example syringe pump system 800 includes a syringe barrel 1, a syringe plunger 2, a plunger drive transmission member 3, a plunger shaft 4, a drive motor assembly 5, and a battery 10. In addition, syringe pump system 800 includes an infusion tubing 6 connected to the syringe housing and a cannula needle 7 for interfacing with the patient. Typically, the syringe pump system 800 may perfuse a drug into the patient, by adding a drug solution into the syringe barrel 1, and inserting the filled syringe into the pump device. The infusion tubing 6 is connected to the syringe at syringe outlet 11. By pushing against the syringe plunger 2 toward the syringe outlet 11, the infusion tubing 6 is filled with the drug, thereby eliminating air from the infusion tubing 6. Once the infusion tubing 6 is completely filled (also called “priming”), the cannula needle 7 can be connected to the patient (usually by inserting the cannula needle 7 into the patient's vein to establish a connection to the patient's blood circulation).

Once the patient is connected to the syringe pump system 800, the perfusion can be started by using the buttons 8 on the device. The display 9 informs about the chosen flow rate and the state of the syringe pump system 800. Once an infusion is started, the drive motor assembly 5 operates to drive the plunger drive transmission member 3 to push the syringe plunger 2. Accordingly, the syringe plunger 2 drives the drug out of the syringe barrel 1 and into the infusion tubing 6. Via the tubing 6 and eventually the cannula needle 7, the drug is perfused into the patient's bloodstream.

When the syringe pump system 800 is used to deliver very low flow rates (and such very low flow rates are typical in applications where syringe pump devices are used), the start-up delay between entering the command to start the infusion using the buttons 8 and the drug reaching the bloodstream of the patient can be significant (e.g., from 30 minutes up to several hours). One reason for this start-up delay is attributable to compliances (i.e., mechanical inaccuracies, system hysteresis, material deflection, etc.) of the conventional syringe pump system. Such compliances are schematically depicted in FIG. 20.

A first compliance is caused by the tolerance between the gears (e.g., gear backlash) of drive motor assembly 5 in the system (refer to section I of FIG. 20). Once the drive motor drives the gears, a certain distance, respectively angle, has to be overcome for the gears to come into force fit and ultimately driving the shaft with an artifice to push the syringe plunger.

A second compliance is caused by mechanical deflections of the plunger 2 and the syringe barrel 1 materials (refer to section II of FIG. 20). The movement of the plunger drive transmission member 3 to push the syringe plunger 2 results in a pressure increase, which ultimately leads to the perfusion of the drug. In the midterm, before the drug is perfused, the syringe barrel 1 can be inflated and the syringe plunger 2 can be compressed. While those effects may seem small from some perspectives, for the very low flow rates of the syringe pump devices, those effects can cause a critically disadvantageous delay of drug perfusion.

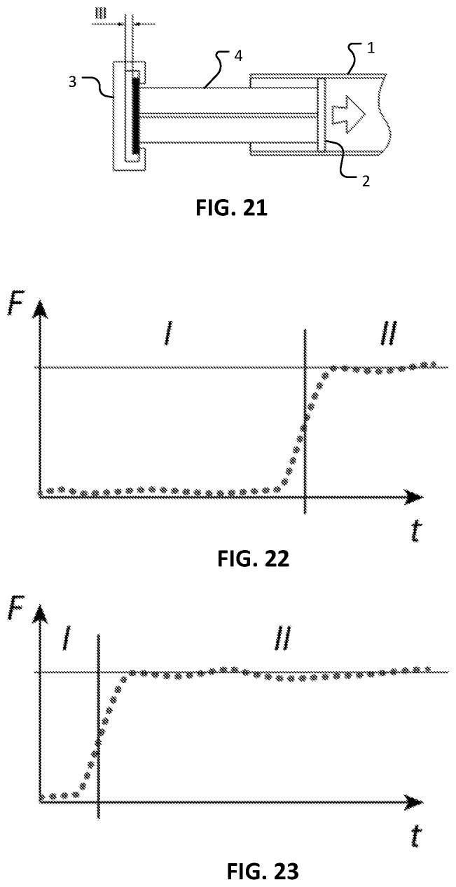

A third compliance is caused by mechanical dimensional tolerances associated with the interface between the plunger drive transmission member 3 and the plunger shaft 4 (refer to section III of FIG. 21). This interface transfers the forces that causes the translation of the plunger shaft 4 and plunger 2. Since direct contact of the plunger drive transmission member 3 and the plunger shaft 4 is not necessarily established when the perfusion system is started, there can be a time delay until the drug delivery actually starts. Additionally, if the syringe pump is above the patient, the plunger 2 can sometimes be pulled toward the patient because of the resulting pressure difference associated with the height difference between syringe pump and patient. Therefore, the plunger drive transmission member 3 can be designed with a latch that prevents the movement of the plunger 2 into the direction of the syringe outlet 11 and a subsequent uncontrolled emptying of the syringe into the patient. The plunger drive transmission member 3 featuring a latch with the plunger in the end position are depicted in FIG. 21. In many syringe pump designs, there is a tolerance in the latch design, which results in a gap between the plunger drive transmission member 3 and the latch itself. This gap is another source of compliance for the syringe pump system 800.

A fourth compliance is attributable to the expansion of the material of the infusion tubing 6 (refer to section IV of FIG. 20). A pressure increase will lead to an inflating of the tubing 6 prior to a drug perfusion and could cause a delay in the drug delivery to the patient. This fourth compliance is the least significant of the four elasticities.

In order to compensate for, or to reduce the effects of, the four compliances described above, a flow sensor 12 such as any of those described herein can be included in syringe pump system 800. By including a flow sensor 12 capable of detecting and quantifying the low flow inside the infusion tubing, and providing a measurement of the flow after the syringe housing, the information about the flow after the syringe housing can be used to reduce the time in the start-up delay and increase the speed of delivering the drug to the patient.

In one embodiment, the flow sensor 12 comprises a heating element and a single temperature sensor as described above. The flow sensor 12 is configured to abut the perfusion tubing while being separable therefrom (not affixed to the tubing). The temperature sensor is positioned to measure the temperature of the fluid within the perfusion conduit. The heater is wired to keep at a constant level above the fluid temperature and the voltage drop across the heater is used to measure the flow rate within the perfusion conduit.

In another embodiment, the flow sensor 12 comprises a heating element and two temperature sensors, up-stream and downstream from the heating element as described above. The temperature difference between the two temperature sensors is used to measure the flow rate within the perfusion conduit. The flow sensor 12 is configured to abut the perfusion tubing while being separable therefrom (not affixed to the tubing).

In yet another embodiment, the flow sensor 12 is a multi-model flow sensor, comprising a heating element and three temperature sensors, and operating at two flow-rate-sensing-modes as described above. The flow sensor 12 is configured to abut the perfusion tubing while being separable therefrom (not affixed to the tubing).

In one embodiment, the flow sensor 12 is designed to abut the infusion tubing from the outside of the infusion tubing. In another embodiment, the flow sensor 12 is designed to abut a special interface section of the infusion tubing which comprises a flow channel, covered by a thin membrane. In yet another embodiment, the flow sensor 12 is designed to abut the syringe at the outlet of the syringe. In one embodiment, the outlet of the syringe is designed to comprise a flow channel, covered by a thin membrane.

For the following control of the flow rate of the perfusion, both a calibrated signal from the flow sensor 12 (e.g., carried out during the priming of the infusion tubing 6) in absolute units (e.g. ml/h) or an uncalibrated signal in e.g. sensor voltage might be used. At the standstill of the system, the flow at the sensor is zero, and the sensor signal is at an according value. After the command to start the infusion is entered at the buttons 8, the engine starts to drive the gears, which drives the plunger drive transmission member 3 to push the syringe plunger 2, which inflates the syringe barrel 1 and ultimately drives the drug past the flow sensor 12. This change in the flow rate is detected by a change in the signal of the flow sensor 12, regardless if a calibrated or an uncalibrated sensor signal is used. This change in the sensor signals signifies the start of the drug delivery into the tubing 6 and thus, approximately, the start of the drug delivery into the bloodstream of the patient. By creating a feedback from the sensor 12 signal to the drive motor assembly 5 control, the start-up delay of the syringe pump can be reduced significantly. A drug delivery procedure, making use of the sensor 12 signal is described in the following paragraph.

Since the diameter of the syringe barrel 1 is a known parameter, as is the translation from the drive motor assembly 5 motion to the plunger 2 movement, the rotational speed of the drive motor assembly 5 is used to control the rate of the drug delivery. For example, if the plunger 2 of a syringe with a diameter of 10 mm is displaced by 1 mm, approximately 78.5 microliters are perfused. If, e.g., one full rotation of the drive motor assembly 5 translates to 1 mm displacement of the plunger drive transmission member 3 to push the syringe plunger 2, on rotation of the drive motor assembly 5 per minute equals 78.5 microliters per minute, which equals 4,712 microliters per hour, or 4.7 ml/h. If the user enters this flow rate, normally the drive motor assembly 5 would start rotating at that speed.

When measuring the flow at a location adjacent to the syringe outlet 11, the device is able to overcome the initial compliance of the syringe pump system 800. That is, when starting the infusion, the drive motor assembly 5 starts rotating at a higher speed, causing the compliance of the system to be removed quicker. When the change in the flow sensor 12 signal is detected (e.g., if a pre-defined threshold of the flow rate is exceeded), the drive motor assembly 5 speed is reduced to one rotation per minute, or whatever flow rate is chosen. The change in the flow rate indicates the overcoming of the compliance, allowing a significantly reduced start-up delay of the drug delivery to the patient. The operability of the flow sensor 12 could be verified during the priming of the infusion tubing.

This relationship is illustrated in FIG. 23 in comparison to FIG. 22. FIG. 22 shows the flow rate during a conventional start-up of a syringe pump device. The compliance during phase I, results in a start-up delay of the flow rate. The rotation speed of the drive motor assembly 5 is constant throughout the whole procedure. In FIG. 23, the drive motor assembly 5 is driven at a higher speed during phase I of the start-up, and reduced once the flow sensor 12 signal signifies an increased flow at the flow sensor 12. Therefore, the start-up delay of the syringe pump system 800, caused by the compliance of the devices as described above, is significantly reduced.

The flow sensor 12 might be designed to abut the infusion tubing 6 (while not being affixed to the infusion tube 6) or a specially designed interface (e.g., including a thin membrane interface). The sensor 12 could also be inside the flow channel, or fixed to the flow channel. The sensor 12 can be integrated in any way that allows a measurement of the flow signal at the exit 11 of the syringe barrel 1, e.g. via a thermal coupling of the sensor elements to the flow channel.

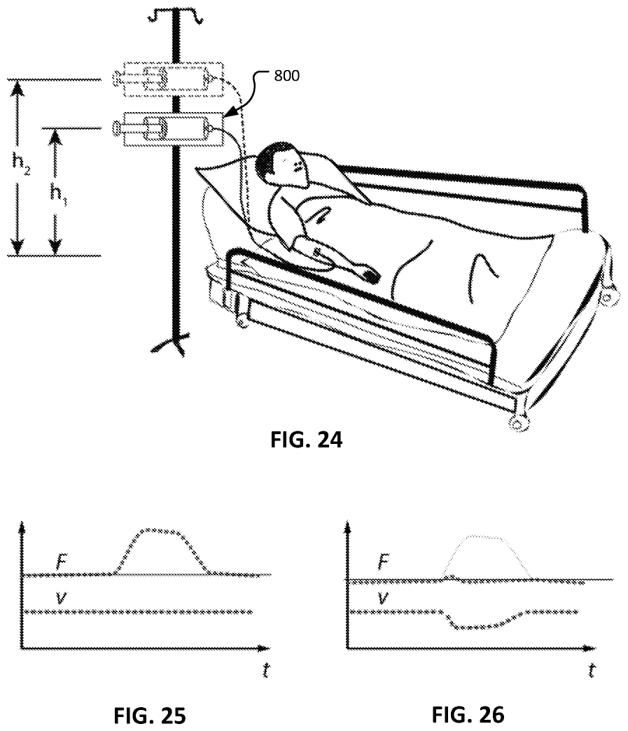

Referring also to FIGS. 24-26, another effect of the compliance in the syringe pump system 800 happens if the height of the syringe pump device is changed (e.g., when the syringe pump is put in into a different slot of a pump rack containing several infusion pumps). As depicted in FIG. 24, there is a height difference (h₂ minus h₁) between the syringe pump device and the patient. This height difference, even if it is negative, causes a certain pressure difference in the therapeutic medical fluid. If the height is changed, also the pressure difference between the pump and the patient is changed. Due to the compliance of the syringe pump system 800, this pressure change results in a change of the pressure equilibrium in the system. For example, if the pump height is increased, the pressure difference between pump and patient is increased. In this case, the drug is pulled out of the syringe, resulting in a bolus delivery to the patient, as illustrated in FIG. 25. This bolus delivery can be critical. The same effect can happen if the pump is lowered, however then there would be a drop in the delivered drug or even blood pulled out of the patient.

In the current invention, a flow sensor 12 at the exit 11 of the syringe barrel 1 can measure the drug flow. Again, this measurement could be calibrated or uncalibrated. The flow sensor 12 signal is fed back to a control unit, which controls the rotation of the drive motor assembly 5. If the flow sensor 12 detects an increase in the flow rate (e.g., if the signal crosses an upper threshold), the control unit regulates the drive motor assembly 5 rotation accordingly to keep the flow rate at a constant level. During the duration, in which the bolus delivery would normally occur, the drive motor assembly 5 turns at a lower speed than normal. After the duration in which the bolus delivery would normally occur, the drive motor assembly 5 speed would return to the normal level. This is illustrated in FIG. 26, where “F” denominates the flow rate measured by the sensor 12, and “V” denominates the speed of the drive motor assembly 5.

The same principle can be applied to height changes associated with a lowering of the height of the syringe pump. The decrease in the flow rate would be detected by the flow sensor 12, causing the drive motor assembly 5 to rotate at a higher speed. and preventing a drop in the perfusion rate to the patient.

Both the start-up delay compensation and the height-change bolus compensation could be counteracted by controlling the drive motor assembly 5 speed to prevent excessive motor speeds or an overshooting of the syringe pump system 800.

The signal from the flow sensor 12, used to control the drive motor assembly 5 speed could either be the complete history of the signal, including measurements done at the priming of the infusion tubing. It could also be a “moving window,” in which a limited number of recent measurements are used for the control. It could also be a combination of any measurements done during the operation of the syringe pump (e.g., measurements during priming and a limited number of recent measurements).

Additional to the absolute/relative value of the flow sensor 12 signal, a threshold can be defined. When the signal crosses this threshold, an action is triggered. This threshold can be either an upper or a lower threshold, or a combination of both an upper and a lower threshold. Additional to the signal, or the threshold of the signal, the first derivation of the signal, i.e., the slope of the signal, can be used to trigger control actions of the drive motor assembly 5.

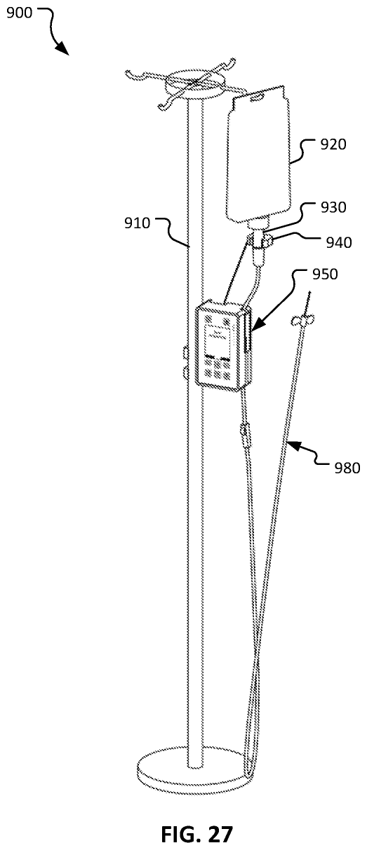

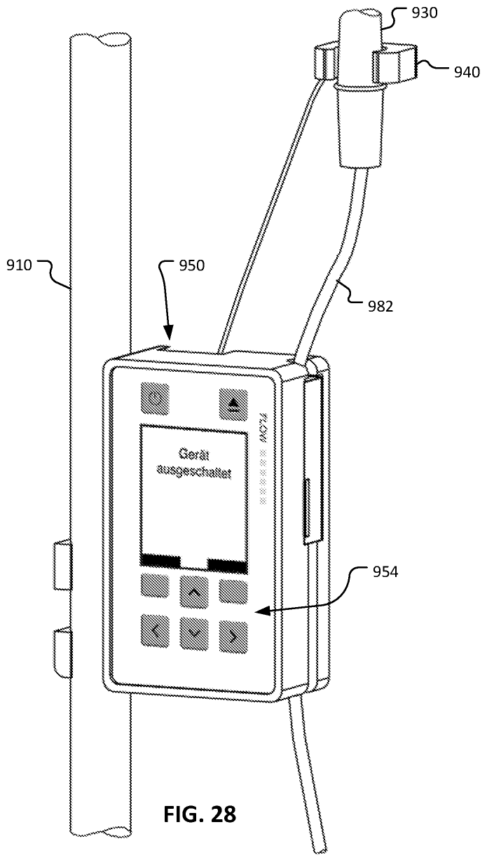

Referring to FIGS. 27 and 28, an example infusion system 900 includes a bottle rack 910 (or similar structural support(s)), a therapeutic medical fluid reservoir 920 (e.g., a bag, bottle, etc.), a drip chamber 930, a drop counter 940, an infusion control system 950, and an infusion tube set 980. In the depicted arrangement, the infusion control system 950 is releasably mounted to a pole of the bottle rack 910. The therapeutic medical fluid flows from the reservoir 920, to the drip chamber 930 (where the therapeutic medical fluid flows drop-by-drop), and into the tube 982 of the infusion tube set 980 which is releasably coupled with the infusion control system 950.

The infusion control system 950 can be used to control the flow rate of the therapeutic medical fluid to a set point that is established by a user of the infusion system 900. The set point can be entered into the infusion control system 950 via a user interface 954 of the infusion control system 950. Thereafter, a control valve of the infusion control system 950 can adjustably pinch the tube 982 of the infusion tube set 980 to modulate the flow of the therapeutic medical fluid to the set point.

A flow sensor device (such as any of the flow sensor devices described herein that abut but are not affixed to the tube 982) can be included in the infusion control system 950. The flow sensor device can provide an accurate indication of the actual flow rate of the therapeutic medical fluid so as to facilitate closed-loop control of the flow rate. In some embodiments, the drip chamber 930 and the drop counter 940 can be used to calibrate the flow sensor device.

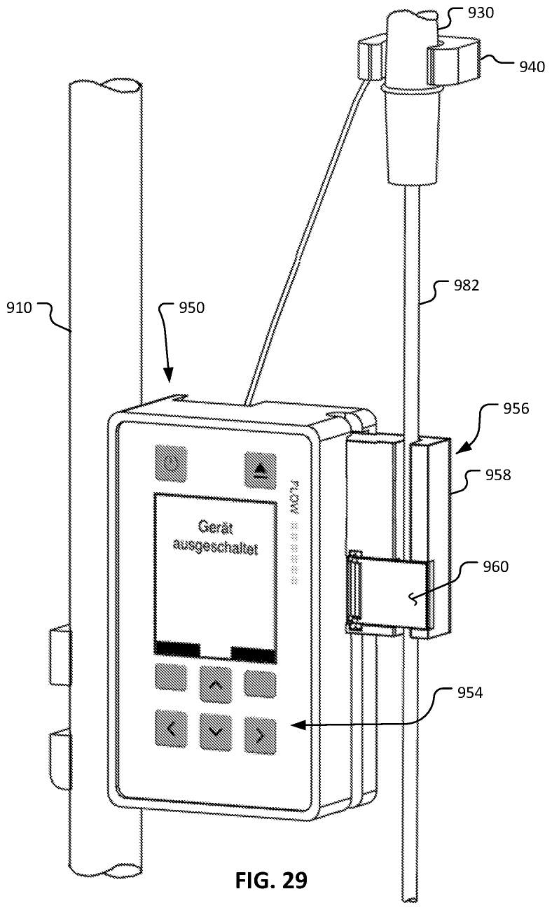

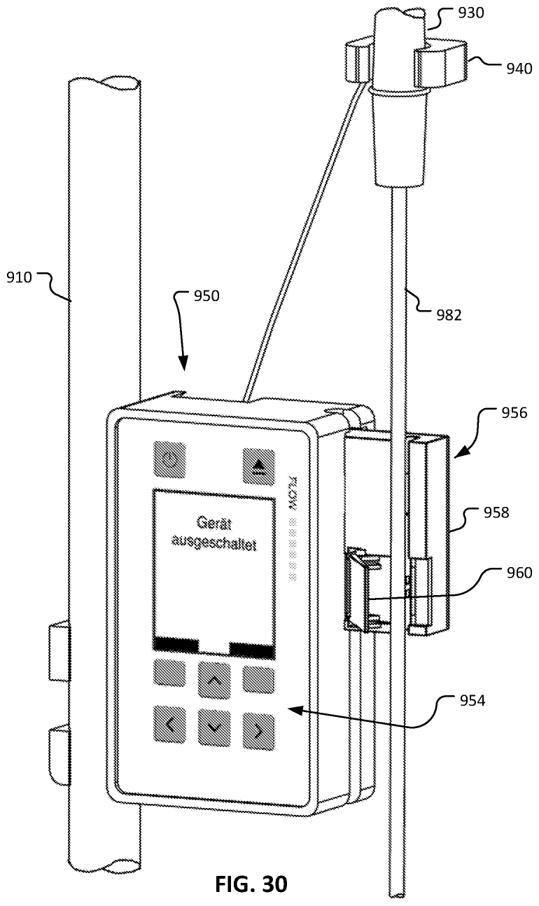

Referring also to FIGS. 29 and 30, the tube 982 of the infusion tube set 980 can be releasably coupled to the infusion control system 950. For example, in the depicted embodiment, the infusion control system 950 includes a drawer assembly 956 that releasably couples with the tube 982. The drawer assembly 956 can be translated laterally outward (as shown in FIGS. 27 and 28) so that the tube 982 can be conveniently coupled to, or uncoupled from, the infusion control system 950. In the normal operating mode of the infusion control system 950, the drawer assembly 956 (with the tube 982 coupled thereto) is positioned within the housing of the infusion control system 950 (as shown in FIGS. 25 and 26).

In the depicted embodiment, the drawer assembly 956 includes a tube engagement member 958 and a tube retainer door 960. The tube engagement member 958 defines a channel that releasably receives the tube 982.

The tube retainer door 960 is pivotably attached to the tube engagement member 958. When loading the tube 982 into engagement with the drawer assembly 956, the tube retainer door 960 is opened as shown in FIG. 28 so that the tube 982 can be positioned within the entire length of the channel defined by the tube engagement member 958. Thereafter, the tube retainer door 960 can be pivoted closed and latched in relation to the tube engagement member 958. When the tube retainer door 960 is closed to detain the tube 982, the tube retainer door 960 presses the tube 982 against a flow sensor within the infusion control system 950.

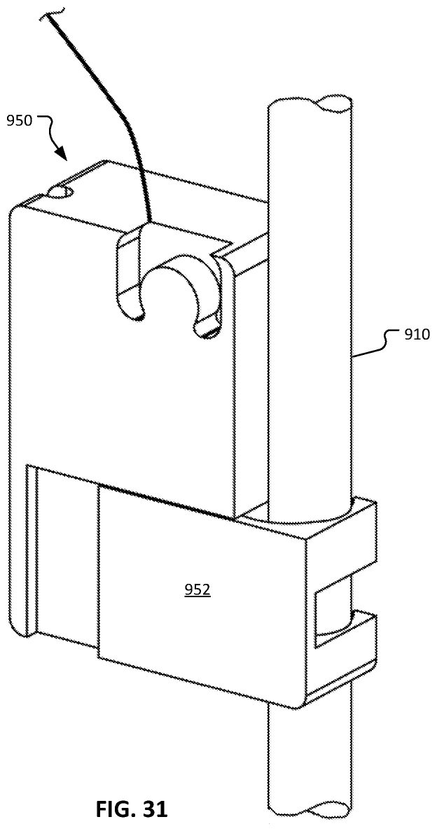

Referring to FIG. 31, in the depicted embodiment, the infusion control system 950 includes a slidable clamp mechanism 952 that can releasably couple the infusion control system 950 to the bottle rack 910. In some embodiments, the slidable clamp mechanism 952 is spring biased so as to provide a clamping force to squeeze the bottle rack 910 in the arrangement as shown.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

It is very important to understand that one or more features from a particular device, system, or method described herein can be combined with one or more features from one or more other devices, systems, or methods described herein. Moreover, without limitation, all such combinations and permutations are within the scope of this disclosure.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1-17. (canceled)

18. A control device for controlling a flow rate of a perfusion liquid flowing through a perfusion conduit, the control device comprising:
a flow rate sensor configured to abut against the perfusion conduit, the flow rate sensor not fixed to the perfusion conduit, the flow rate sensor comprising:
a first temperature sensor;
a second temperature sensor;
a third temperature sensor, wherein the second temperature sensor is disposed between the first temperature sensor and the third temperature sensor; and
a heating element disposed between the first and second temperature sensors;
a regulating valve configured to adjustably compress the perfusion conduit to regulate the flow rate of the perfusion liquid flowing through the perfusion conduit; and
a control unit in communication with the flow rate sensor and the regulating valve, the control unit configured to determine the flow rate of the perfusion liquid in a first flow-rate-sensing-mode using a difference in temperatures detected by the first and second temperature sensors, the control unit configured to determine the flow rate of the perfusion liquid in a second flow-rate-sensing-mode based on a detected voltage drop across the heating element while the voltage supplied to the heating element is equilibrated with the voltage drop across the third temperature sensor,
the control unit configured to adjust the regulating valve based on the determined flow rate of the perfusion liquid.

19-21. (canceled)

22. The control device of claim 18, wherein the control device is configured to releasably couple with the perfusion conduit such that, while the perfusion conduit is coupled with the control device, the flow rate sensor: (i) abuts against the perfusion conduit while the control device is arranged in a first configuration, and (ii) is spaced apart from the perfusion conduit while the control device is arranged in a second configuration.

23. The control device of claim 18, wherein a movable portion of the control device is configured to releasably couple with the perfusion conduit and to: (i) position the perfusion conduit in contact with the flow rate sensor in the first configuration and (ii) position the perfusion conduit separated away from the flow rate sensor in the second configuration.

24. The control device of claim 23, wherein the movable portion of the control device comprises a portion of the regulating valve that is configured to adjustably compress the perfusion conduit.

25. The control device of claim 18, wherein the flow rate sensor is configured to abut against a round outer wall of a standard tubing portion of the perfusion conduit.

26. The control device of claim 18, further comprising a drop counter in communication with the control unit, and wherein the control unit is configured to use the drop counter to calibrate the flow rate sensor.

27. The control device of claim 18, further comprising a slidable clamp mechanism by which the control device can be releasably coupled to the pole.

28. The control device of claim 18, wherein the control device is configured to adjust the regulating valve to modulate the flow rate to a set point.

29. The control device of claim 28, further comprising a user interface, and wherein the set point can be entered using the user interface.

30. The control device of claim 18, further comprising a drawer assembly that releasably couples the perfusion conduit to the control device.

31. The control device of claim 30, further comprising a housing containing the control unit, and wherein the drawer assembly can be: (i) positioned within the housing and (ii) translated laterally outward from the housing.

32. The control device of claim 30, wherein the drawer assembly defines a channel that releasably receives the perfusion conduit.

33. A multi-modal flow rate sensor comprising:
a heating element;
a first temperature sensor disposed at a first side of the heating element;
a second temperature sensor disposed at a second side of the heating element, the second side of the heating element being opposite of the first side of the heating element; and
a third temperature sensor disposed at the second side of the heating element,
wherein the multi-modal flow rate sensor is operable in (i) a first flow-rate-sensing-mode and (ii) a second flow-rate-sensing-mode that differs from the first flow-rate-sensing-mode,
wherein the third temperature sensor is not used for first flow-rate-sensing-mode, and wherein the first and second temperature sensors are not used for the second flow-rate-sensing-mode.

34. The flow rate sensor of claim 33, wherein the flow rate sensor is configured to abut against tubing while not fixed to the tubing and to measure a flow rate of a fluid flowing in the tubing, and wherein the heating element is configured to heat the fluid flowing in the tubing.

35. The flow rate sensor of claim 33, wherein the first flow-rate-sensing-mode also allows for determining a flow direction of a fluid.

36. The flow rate sensor of claim 33, wherein the first flow-rate-sensing-mode is better suited for measuring low flow rates than the second flow-rate-sensing-mode.

37. The flow rate sensor of claim 33, wherein the flow rate sensor is contained on a single flow sensor chip.
